(19) United States
(12) Patent Application Publication
(10) Pub. No.: US 2016/0022618 A1
(43) Pub. Date: Jan. 28, 2016

(54) 4-HYDROXY BENZOATE DERIVATIVES FOR USE IN THE TREATMENT OF INFECTION, INFLAMMATION OR PAIN

(71) Applicant: Roy Arlington Jordan, Surrey (GB)

(72) Inventor: Roy Arlington Jordan, Surrey (GB)

(21) Appl. No.: 14/809,454

(22) Filed: Jul. 27, 2015

Related U.S. Application Data
(63) Continuation-in-part of application No. 14/322,333, filed on Jul. 2, 2014, now Pat. No. 9,090,555.
Application No. 14/322,333 is a continuation of application No. 12/663,451, filed on May 25, 2010, now Pat. No. 8,785,504.

(30) Foreign Application Priority Data
Jun. 20, 2007 (GB) 0711947.2

(51) Int. Cl. A61K 31/192; A61K 33/02

(52) U.S. Cl. CPC A61K 31/192; A61K 33/02

(57) ABSTRACT

A method is described for treatment of a human in need of repeated applications, over a period of time, of smelling salts to provide nasal inhalation of ammonia gas. The method comprises administration to the human as a nasal spray of a therapeutically effective amount of a pharmaceutical composition comprising triethanolammonium 4-hydroxybenzoate dissolved in an aqueous solution within a time interval preceding each application of smelling salts during which the nasal spray remains effective to alleviate or prevent pain to the nasal mucosa caused by the subsequent application of smelling salts.

### PRIORITY CLAIM

The present application is a continuation-in-part of U.S. patent application Ser. No. 14/322,333, filed Jul. 2, 2014, which is a continuation of U.S. patent application Ser. No. 12/663,451, filed May 25, 2010 issued as U.S. Pat. No. 8,785,504 on Jul. 22, 2014, which claims priority to PCT/GB2008/002001 filed Jun. 12, 2008 which claims priority to UK Application No. 0711947.2, filed Jun. 20, 2007.

### BACKGROUND OF THE INVENTION

1. Field of the Invention

The invention generally provides compounds and compositions for use in the topical treatment of infection, inflammation or pain, a method of preparing the compounds, uses of the compounds and compositions in the preparation of medicaments for the topical treatment of infection, inflammation or pain, and a method of topical treatment or alleviation of infection, inflammation or pain using the compounds and compositions.

2. Description of the Relevant Art

There are many common human or animal conditions which result in infection, inflammation and/or pain for which there are limited or no effective preventative or curative treatment. These include allergic and non-allergic conditions such as eczema, acne, psoriasis, geographic tongue and adverse reactions to insect bites (e.g., flea or midge bites). In some instances, steroidal treatments may be applied to the skin of a patient to treat these conditions with limited success and often with adverse side effects.

Smelling Salts are chemical compounds used for arousing consciousness. The principal component is usually Ammonium Carbonate, (NH₄)₂CO₃, which may also be provided as an aqueous solution, generally provided in a flask or bottle from which a lid or stopper is removed, or from which a sponge soaked in the solution may be removed, to allow a human in need of such treatment to inhale Ammonia, NH₃, from the salt or from an aqueous solution thereof or a solution in vinegar or alcohol. Perfume may be added, as may other inhalants such as lavender oil or eucalyptus oil. The term “smelling salts” as used herein encompasses all and any of these compositions and formulations. Smelling salts may restore or enhance consciousness and mental alertness, and have long been used in sports medicine and for the alleviation of faintness, for example in pregnant women. Inhalation of NH₃ from smelling salts may also increase mental alertness in patients suffering from other conditions that affect the mental faculties such as dementia.

Ammonia inhaled from smelling salts triggers an inhalation reflex by irritating the nasal mucosa, and elevates the heart rate, blood pressure and brain activity. Repeated use of smelling salts is painful to the nasal mucosa.

A way of ameliorating the above-identified problems has been sought.

### SUMMARY OF THE INVENTION

Reference may be made to my aforesaid U.S. Pat. No. 9,090,555, issued Jul. 28, 2015 and

U.S. Pat. No. 8,785,504, issued Jul. 22, 2014, the whole disclosures of each of which are incorporated herein, for description of compounds of the general formula (I), and more specifically triethanolammonium 4-hydroxybenzoate (TEAB), and their use in the topical treatment of infection, inflammation and/or pain:

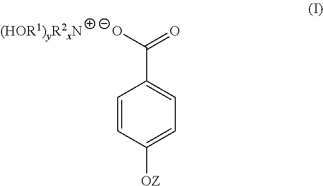

wherein R¹ independently represents a methylene group, an ethylene group or a straight or branched chain C₃ to C₆ alkylene group; R² independently represents a hydrogen atom, a methyl group, an ethyl group or a straight or branched chain C₃ to C₂₀ alkyl group; x represents 0 or an integer from 1 to 4 and y represents 0 or an integer from 1 to 4, wherein the sum of x and y is 4; and Z represents a hydrogen atom or (HOR¹)_{y}R²_{X}N⁺. Compositions including the compound of general formula (I); use of the compound of general formula (I)in the manufacture of a medicament; and methods of medical treatment including the topical application of the compound of general formula (I) are also described in my aforesaid U.S. Patent Applications.

In a first aspect of the present disclosure, a method is provided for treatment of a human in need of repeated applications, over a period of time, of smelling salts to provide nasal inhalation of ammonia gas, the method comprising administration to the human as a nasal spray of a therapeutically effective amount of a pharmaceutical composition comprising triethanolammonium 4-hydroxybenzoate dissolved in an aqueous solution within a time interval preceding each application of smelling salts during which the nasal spray remains effective to alleviate or prevent pain to the nasal mucosa caused by the subsequent application of smelling salts.

Triethanolammonium 4-hydroxybenzoate is preferably present in the aqueous solution in an amount of 0.2 to 5% by weight of the composition, more preferably present in the aqueous solution in an amount of 0.25 to 4% by weight of the composition, and most preferably present in the aqueous solution in an amount of 0.3% by weight of the composition.

In a second and alternative aspect of this disclosure, there is provided, in combination, for the treatment of a human in need of ammonia gas inhalation to enhance brain activity, a supply of smelling salts for application to the human for nasal inhalation of ammonia, and a nasal spray comprising an aqueous solution of triethanolammonium 4-hydroxybenzoate for administration to the human's nose before each application of smelling salts to alleviate or prevent pain caused by the subsequent application of smelling salts.

According to a third alternative aspect of the present disclosure, a method of enhancement of brain activity in a human, comprises the steps in order of:
(1) administration to the human as a nasal spray of a therapeutically effective amount of a pharmaceutical composition comprising triethanolammonium 4-hydroxybenzoate dissolved in an aqueous solution;
(2) application to the human of smelling salts for nasal inhalation of ammonia gas; and
(3) optionally repeating step (1) followed by step (2) at least once.

The nasal spray may be provided in an atomizer.

The embodiments are illustrated by the following Examples which are not intended to limit the scope of the claims.

In example 24, a TEAB composition was tested in vivo for efficacy when used in conjunction with smelling salts. No side effects were observed as a result of the administration of a TEAB composition in the in vivo tests.

### EXAMPLE 1

### Preparation and Characterization of Triethanolammonium 4-hydroxybenzoate (TEAB)

4-hydroxybenzoic acid (10.6 g, 0.08 mol) was dissolved in acetone (200 mL). To the 4-hydroxybenzoic acid solution was added a solution of triethanolamine (9.23 g, 0.07 mol) dissolved in a water (70 mL)/acetone (200 mL) mixture yielding a clear solution. The solution was stirred at room temperature for 1 hour. The solution was stripped of acetone on a rotary evaporator to yield 91 g (100%) of a light yellow solution with n20d of 1.3890. This light yellow solution was found to contain 30% by weight of TEAB in water. A sample of the light yellow solution was reduced to near dryness yielding a white crystalline solid identified as TEAB containing 0.3 moles of water per mole of solid (93-94 C).

To establish the ionic nature of TEAB, 10 g of the light yellow TEAB solution was blended with water (300 mL) and Dowex MB-3 ion exchange resin (Dow Chemical Company) and left to stand overnight. At this time, the liquid component of the blend was separated from the resin and reduced to dryness resulting in no residue. This indicates that the anionic and cationic component of TEAB had been retained by the ion exchange resin and that the solution only contained TEAB.

Elemental analysis (C₁₃H₂₁NO₆.0.3H₂O): Calculated (%) C 53.3, H 7.4, N 4.8, O 34.4. Found (%) C 53.3, H 7.5, N 4.5, O 34.7. ¹H NMR (D₂O): δ 3.4 (—CH₂—), 3.9 (—CH2—), 4.8 (NH), 4.8 (OH), 6.9 (ArH), 7.8 (ArH). Infrared spectroscopy, cm⁻¹: 3450-3600 (v(OH)), 2854 (v(NH)), 1463 (v(C═O)), 1377 (v(C-O)). n20d (50% wt solution of TEAB in water) 1.4482. pH (50% wt solution of TEAB in water) 6.61. Calculated heat of reaction (kJ/mol) −12.0.

### EXAMPLE 2

### Preparation of Triethanolammonium 4-hydroxybenzoate (TEAB)

Triethanolamine (149 g, 1 mol) was dissolved in water (287 mL). To this solution was added solid 4-hydroxybenzoic acid (138 g, 1 mol) over a period of 3 to 4 minutes. As the solid 4-hydroxybenzoic acid was added it reacted and dissolved increasing the temperature of the reaction mixture 5° C. After overnight standing, 573 g (100%) of a light yellow TEAB solution was recovered with n20d of 1.4484 and pH of 6.61. 40 g of the light yellow TEAB solution was reduced to 23 g on a rotary evaporator and this solution poured into acetone (300 mL). After 72 hours, a white crystalline solid was filtered off, washed with acetone and oven dried at 60° C. (m.p. 93-94 C). Yield 19 g.

### EXAMPLE 3

In the following Example, the preparation of two compositions including TEAB for use in the topical treatment of pain are described.

### Composition 1

3 g of TEAB was dissolved in 97 mL of water. This resulted in a composition with 3% by weight of TEAB.

### Composition 2

3 g of TEAB was dissolved in 997 mL of water. This resulted in a composition with 0.3% by weight of TEAB.

### EXAMPLE 4

### Pain Associated with Regular Use of Smelling Salts

In the following Example, composition 2 was tested in conjunction with smelling salts for efficacy for preventing or reducing the pain associated with regular use of smelling salts. A male subject experiencing nasal pain and discomfort as a result of regular use (4-5 doses per day) of smelling salts was treated with composition 2. Treatment was by nasal spray with a single 0.25 to 0.5 mL dose of the solution sprayed as a mist into each nostril immediately before a dose of smelling salts was taken as normal, and no pain was noticed.

Thus, nasal administration of a TEAB composition has shown effect in relieving the pain associated with regular use of smelling salts.

The Example was repeated with a TEAB aqueous composition having a lower concentration of TEAB, namely a 0.25 by weight. Again there no pain was noted when smelling salts were applied immediately afterwards to allow nasal inhalation of ammonia gas.

The percentage by weight of TEAB may be increased, since the pain alleviation effect of TEAB will be maintained at higher concentrations and in larger doses, but compositions with a concentration by weight of more than 5% are wasteful of TEAB. Since the functional effect of smelling salts is by irritation of the nasal mucosa, the nasal spray should not be administered in such great amounts as to interfere with the function of the smelling salts.

While application of smelling salts immediately after application of the nasal spray is preferred, I have found that a few minutes' delay (up to a maximum of 5 minutes, so that the nasal spray does not dry in the nose) is still effective.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Changes may be made in the elements described herein without departing from the spirit and scope of the invention as described in the following claims.

In particular, the nasal spray for use in conjunction with smelling salts as described above may comprise additional ingredients without deviating from the spirit of the invention. By way of non-limiting examples these may include additives to improve the shelf life of the spray or additives to improve the odor of the spray.

What is claimed is:

1. A method of treatment of a human in need of repeated applications, over a period of time, of smelling salts to provide nasal inhalation of ammonia gas, the method comprising administration to the human as a nasal spray of a therapeutically effective amount of a pharmaceutical composition comprising triethanolammonium 4-hydroxybenzoate dissolved in an aqueous solution within a time interval preceding each application of smelling salts during which the nasal spray remains effective to alleviate or prevent pain to the nasal mucosa caused by the subsequent application of smelling salts.

2. The method of claim 1, wherein triethanolammonium 4-hydroxybenzoate is present in the aqueous solution in an amount of 0.2 to 5% by weight of the composition.

3. The method of claim 1, wherein triethanolammonium 4-hydroxybenzoate is present in the aqueous solution in an amount of 0.25 to 4% by weight of the composition.

4. The method of claim 1, wherein triethanolammonium 4-hydroxybenzoate is present in the aqueous solution in an amount of 0.3% by weight of the composition.

5. A kit for the treatment of a human in need of ammonia gas inhalation to enhance brain activity, comprising: a supply of smelling salts for application to the human for nasal inhalation of ammonia; and a nasal spray comprising an aqueous solution of triethanolammonium 4-hydroxybenzoate for administration to the human's nose before each application of smelling salts to alleviate or prevent pain caused by the subsequent application of smelling salts.

6. The kit of claim 5, wherein triethanolammonium 4-hydroxybenzoate is present in the aqueous solution in an amount of 0.2 to 5% by weight of the composition.

7. The kit of claim 5, wherein triethanolammonium 4-hydroxybenzoate is present in the aqueous solution in an amount of 0.25 to 4% by weight of the composition.

8. The kit of claim 5, wherein triethanolammonium 4-hydroxybenzoate is present in the aqueous solution in an amount of 0.3% by weight of the composition.

9. A method of enhancement of brain activity in a human, comprising:
(1) administration to the human as a nasal spray a therapeutically effective amount of a pharmaceutical composition comprising triethanolammonium 4-hydroxybenzoate dissolved in an aqueous solution;
(2) application to the human of smelling salts for nasal inhalation of ammonia gas.

10. The method of claim 9, further comprising repeating step (1) followed by step (2) at least once.
